(19) United States
(12) Patent Application Publication
(10) Pub. No.: US 2004/0259936 A1
(43) Pub. Date: Dec. 23, 2004

(54) Treatment of neoplasia

(76) Inventors: Leonard C Nagarkatti, Richmond, VA (US); Prakash Nagarkatti, Richmond, VA (US); Robert McKallip, Richmond, VA (US); Catherine Lombard, Richmond, VA (US); Seongho Ryu, Richmond, VA (US)

Correspondence Address: NIXON & VANDERHYE, PC, 1100 N GLEBE ROAD, 8TH FLOOR, ARLINGTON, VA, 22201-4714, US

(21) Appl. No.: 10/497,911

(22) PCT Filed: Dec. 9, 2002
(86) PCT No.: PCT/US02/39310

Related U.S. Application Data
(60) Provisional application No. 60/336,732, filed on Dec. 7, 2001.

(51) Int. Cl. A61K 31/353

(52) U.S. Cl. 514/453

(57) ABSTRACT

A method of treating a patient in need of therapy for an abnormality of cells of the immune system is provided comprising administration of a therapeutically effective dose of a compound having CB2 cannabinoid receptor activity. The abnormality is particularly a malignancy such as a leukemia or lymphoma.

The present invention relates to the targeting of CB2 cannabinoid receptors as a novel therapy to treat malignant lymphoblastic disease, particularly by adminsitration of active molecules possessing at least some effective CB2 receptor agonist activity to patients sufferring from such disease.

Marijuana is one of the oldest drugs of abuse, although its medicinal value has also been known for several centuries. Delta-9-tetrahydrocannabinol (THC) is the major psychoactive component in marijuana (see reference 1). THC and other synthetic cannabinoids have been used as potential therapeutic agents in alleviating such complications as intraocular pressure in glaucoma, cachexia, nausea, and pain (see reference 2). Interest in the potential medicinal use of cannabinoids grew recently with the discovery of 2 cannabinoid receptors, CB1 and CB2 (references 3 and 4 incorporated herein by reference). CB1 receptors are expressed predominantly in the brain, whereas CB2 receptors are found primarily in the cells of the immune system. Furthermore, endogenous ligands for these receptors capable of mimicking the pharmacologic actions of THC have also been discovered. Such ligands were designated endocannabinoids and include anandamide and 2-arachidonoyl glycerol. (reference 5-7). The physiologic function of endocannabinoids and cannabinoid receptors remains unclear.

Recently, anandamide was shown to inhibit the proliferation of human breast cancer cell lines MCF-7 and EFM-19 in vitro (reference 8). Also, THC was shown to induce apoptosis in human prostate PC-3 cells and in C6 glioma cells in culture (references 9 and 10). THC-induced apoptosis involved cannabinoid receptor-dependent (references 8,11) or -independent pathways (references 9,10). Such studies have triggered interest in targeting cannabinoid receptors in vivo to induce apoptosis in transformed cells. To this end, cannabinoids were shown recently to inhibit the growth of C6 glioma cells in vivo (references 12,13)

The present inventors have noted that cells of the immune system express high levels of CB2 receptors which they considered might be implicated in induction of apoptosis in normal or transformed immune cells. By using both murine and human leukemia and lymphoma lines as well as primary acute lymphoblastic leukemia (ALL) cells they have demonstrated that ligation of CB2 receptors can induce apoptosis in a wide range of cancers of immune-cell origin. Furthermore, they demonstrate that TEC can inhibit the growth of murine lymphoma cells in vivo by inducing apoptosis and, in test experiments, completely cure approximately 25% of the mice bearing that tumor. Current data suggest that CB2 agonists that are devoid of psychotropic effects may constitute a novel and effective modality to treat malignancies of the immune system.

The inventors have particularly found that exposure of murine tumors EL-4, LSA, and P815 to delta-9-tetrahydrocannabinol (THC) in vitro led to a significant reduction in cell viability and an increase in apoptosis. Exposure of BL-4 tumor cells to the synthetic cannabinoid HU-210 and the endogenous cannabinoid anandamide lcd to significant induction of apoptosis, whereas exposure to WINS55212 was not effective. Treatment of EL-4 tumor bearing mice with THC in vivo led to a significant reduction in tumor load, increase in tumor-cell apoptosis, and increase in survival of tumor-bearing mice.

The inventors have examined of a number of human leukemia and lymphoma cell lines, including Jurkat, Molt-4, and Sup-T1, and have determined that they expressed CB2 but not CB1 receptors. These human tumor cells were also susceptible to apoptosis induced by THC, HU-210, anandamide, and the CB2-selective agonist JWH-015. This effect was mediated at least in part through the CB2 receptors because pretreatment with the CB2 antagonist SR144528 partially reversed the THC-induced apoptosis. Culture of primary acute lymphoblastic leukemia cells with THC in vitro reduced cell viability and induced apoptosis. Thus CB2 cannabinoid receptors expressed on malignancies of the immune system are capable of serving as potential targets for the induction of apoptosis. CB2 agonists lack psychotropic effects, they can serve as novel anticancer agents to selectively target and kill tumors of immune origin.

One example of a CB2 specific agonist, JWH-015, has formula (2-Methyl-1-propyl-1H-indol-3-yl)-1-naphthalenylmethanone, having M.W. 327.43. It is soluble to 10 mM in DMSO and to 25 mM in ethanol. This is a selective CB₂ agonist (K, values are 13.8 and 383 nM as measured at human cloned CB₂ and CB₁ receptors expressed in CHO cells). See Griffin et al (1999) Evidence for the presence of CB₂-like receptor on peripheral nerve terminals. Eur. J. Pharmacol. 339 53. Pertwee et al (1999) Pharmacology of cannabinoid receptor ligands. Curr. Med. Chem. 6 635. Chin et al (1999) The third transmembrane helix of the cannabinoid receptor plays a role in the selectivity of aminoalkylindoles for CB2, peripheral cannabinoid receptor. J. Pharmacol. Exp. Ther. 291 837. All these references are incorporated herein by reference.

Other selective CB2 agonists are taught by Wiley et al (incorporated herein by reference—. J Pharmacol Exp Ther 2002 301: 679-689), particular compounds being resorcinols. Preferred selective CB2 agonists for use on the present invention have an affinity for CB2 receptors that is at least five times that for CB1, more preferably at least 10 times, still more preferably at least 20 times and most advantageously 100 or more times.

Thus a first aspect of the present invention provides a method of treating a patient in need of therapy for an abnormality of cells of the immune system comprising adminsitration of a therapeutically effective dose of a compound having CB2 cannabinoid receptor activity.

Preferably the abnormality is a malignancy of the immune system, autoimmune disease, septic shock, transplantation reaction and allergy. Most preferably the abnormality is leukemia or lymphoma, particularly primary acute lymphoblastic leukemia (ALL).

Advantageously, the compound is a CB2 agonist that has reduced psychotrophic activity as compared with classical CB1 receptor agonists such as THC.

Administration of the aforementioned CB2 agonist compounds or a formulation thereof need not be restricted by route. Options include enteral (for example oral and rectal) or parenteral (for example delivery into the nose or lung or injection into the veins, arteries, brain, spine, bladder, peritoneum, muscles or subcutaneous region). The treatment may consist of a single dose or a plurality of doses over a period of time. The dosage will preferably be determined by the physician but may be between 0.01 mg and 1.0 g/kg/day, for example between 0.1 and 500 mg/kg/day. In terms of dose per square meter of body surface, the compound can be administered at 1.0 mg to 1.5 g per m² per day, for example 3.0-200.0 mg/m²/day.

Whilst it is possible for a compound of the invention to be administered alone, it is preferable to present it as a pharmaceutical formulation, together with one or more acceptable carriers and/or excipients. The carrier(s) and/or excipients must be “acceptable” in the sense of being compatible with the compound of the invention and not deleterious to the recipients thereof.

The formulations may conveniently be presented in unit dosage form and may be prepared by any of the methods well known in the art of pharmacy. A unit dosage form may comprise 2.0 mg to 2.0 g, for example 5.0 mg to 300.0 mg of active ingredient. Such methods include the step of bringing into association the active ingredient, i.e. the compound of the invention, with the carrier and/or excipients which constitute one or more accessory ingredients., In general the formulations are prepared by uniformly and intimately bringing into association the active ingredient with liquid carriers or finely divided solid carriers and/or excipients and/or two or all of these, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropyl-methyl cellulose), lubricant, inert diluent, preservative, disintegrant (e.g. sodium starch glycollate, PVP, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which may render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid earner, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient

It should be understood that in addition to the ingredients particularly mentioned above the formulations of this invention may include other agents conventional in the art having regard to the type of formulation in question, for example those suitable for oral administration may include flavouring agents.

In a second aspect of the present invention there is provided the use of a compound of the first aspect of the invention for the manufacture of a medicament for the treatment of abnormalities of the immune system, particularly malignancies of the immune suystem, such as leukemia and lymphoma.

The present inventors have demonstrated that THC and other cannabinoids can induce apoptosis in murine and human leukemia and lymphoma cell lines as well as primary ALL cells. The human tumor-cell lines screened expressed CB2 but not CB1 receptors, whereas the murine tumors expressed both CB1 and CB2 receptors.

Ligation of CB2 receptors is sufficient to induce apoptosis inasmuch as CB2-selective agonists can induce apoptosis in tumor cells. THC-induced apoptosis in human tumor-cell lines is now shown to be reversed by CB2 antagonists. THC was effective not only in vitro but also in vivo, as demonstrated by its ability to induce apoptosis and decrease the tumor load. Moreover, THC treatment could cure approximately 25% of the mice bearing a syngeneic tumor. Thus targeting CB2 receptors on tumor cells of immune origin provides a novel and relatively non-toxic approach to treating such cancers.

The interactions between cannabinoids and their receptors in regulating neurobehavioral functions have been extensively studied. Cannabinoids have also been shown to alter immune functions, although the precise mechanisms remain unclear. Also, the physiologic functions of cannabinoid receptors on immune cells and the role played by endocannabinoids in immune-cell regulation ran unresolved. The inentors have also demonstrated that administration of THC to C57BL/6 mice led to a marked decrease in the cellularity of the thymus and spleen that resulted from the induction of apoptosis in immune cells.

Recently, cannabinoids have been shown to induce apoptosis in tumor cells in vitro. (see references 9,10,12,18,19) Together, such studies suggest the possible use of cannabinoids as anticancer agents. The exact mechanism by which THC induces apoptosis in normal and transformed lymphocytes remains unclear. It is believed that THC and other cannabinoids can act by two distinct mechanisms. Because of its lipophilic properties, it was thought that THC acted through direct intercalation into the cell membrane. However, it was soon realized that the activity of cannabinoids was highly stereospecific, suggesting that the lipophilic properties were not solely responsible for the cannabinoids' activity. Since then, receptors for cannabinoids have been characterized. These receptors share only 44% homology, but most cannabinoids tested show similar binding affinity to both receptors (reference 20).

Both receptors are coupled to G-protein, suggesting that endogenous cannabinoids may play a role in cell signaling (reference 1). Therefore, it is possible that the observed effects of THC on the immune response, including the induction of apoptosis, may be mediated by signals initiated through these receptors. For example, Galve-Roperh et al (reference 12) demonstrated that apoptosis induced by THC in C6 glioma cells in vivo involved a cannabinoid receptor-dependent pathway.

In contrast, others have shown in C6 glioma or a prostate cancer cell model that THC-induced apoptosis was independent of the involvement of the CB1 and CB2 receptors. In the current study, several observations suggested that ligation of the CB2 receptor can induce apoptosis in tumors of immune origin. For example, the human tumor cells such as Jurkat and Sup-T1 expressed only CB2 receptors, and the THC-induced apoptosis in these tumor cells was inhibited at least in part by CB2 antagonists. These studies, however, did not rule out the possibility that ligation of CB1 receptors on murine tumors of immune origin would also induce apoptosis. In fact. The inventors have observed that addition of CB1 antagonist to the EL-4 tumor cells can also inhibit the apoptosis. It should be noted, however, that the cannabinoid receptor antagonists can act as inverse agonists (references 21,22) and thereby prevent apoptosis through an alternate pathway. It is for this reason that the inventors used human cell lines that expressed only the CB2 receptors and showed using CB2 antagonists that ligation of CB2 receptors alone is sufficient to induce apoptosis.

THC is well known for its impact on the cytokine network (reference 23). For example, the presence of THC or activation of the CB1/CB2 receptors can block forskolin-induced accumulation of cyclic adenosine monophosphate (cAMP) (references 24-26) and reduced cAMP levels correlate with the repression of interleukin-2 (IL-2) transcription and secretion (reference 27). IL-2 plays an important role in the regulation of apoptosis (references 28-30). Therefore, reduction in the levels of IL-2 or other cytokines following exposure to THC may partly account for increased apoptosis. The inventors have demonstrated that IL-2 can act as an aurocrine growth factor in the autonomous proliferation of transformed T cells (references 31, 32). Thus, inhibition of IL-2 production by THC could lead to decreased proliferation and apoptotic cell death.

CB1 receptors are expressed in the central nervous system as well as the pituitary gland, immune cells, reproductive tissues, gastrointestinal tissues, heart, lungs, urinary bladder, and adrenals (reviewed by Berdy-shev reference 1). In contrast, CB2 receptors are found primarily in immune cells, including T cells, B cells, natural killer cells, macrophages, neutrophils, and mast cells (references 33,34).

Thus, the selective expression of CB2 receptors on the immune cells provides a unique opportunity to target malignancies of the immune system by using CB2 agonists to induce apoptosis and thereby provide new avenues to treat such cancers The advantage in using CB2-selective agonists also stems from the fact that such a treatment is devoid of the psychotropic effects that are characteristic of CB1 agonists. It should be noted that in the current study, we randomly selected a few murine and human tumor-cell lines of immune origin that were all found to be sensitive to cannabinoid-induced apoptosis.

The dose of THC that induced apoptosis in vitro in the current study was found to be 10 μM or greater using serum-containing medium and 3 μM or higher in serum-free medium. Similar observations were made by others who also noted that THC was less effective in inducing cell death in the presence of serum (reference 17) This is believed to be the result of direct interactions between serum proteins, such as albumin, and cannabinoids (reference 16). The doses of THC used in vitro in the current study were pharmacologically relevant because in an earlier study, rats injected with 50 mg/kg T4C were shown to exhibit 10 μM THC in the serum within 10 hours of administration (reference 35) Also, in these studies, mice were given as high as 500 mg/kg 5 times a week for 2 years. Interestingly, despite such high doses, the survival of dosed rats was higher than in controls. Also, the incidence of a wide range of cancers in mice and rats treated with THC was reduced in a dose-dependent manner (reference 35).

In most previous studies, the effect of THC or other cannabinoids in inducing apoptosis in nonlymphoid tumor-cell lines was seen only after exposure for 2 or more days (references 9,10,12,13). In contrast, in the current study, we were able to demonstrate marked induction of apoptosis in lymphoid tumors as early as 4 hours following culture with THC. These data suggest that lymphoid tumors may be highly sensitive to THC-induced apoptosis. Anandamide was shown recently to induce apoptosis in human neuroblastoma (CHP100) and lymphoma (U937) cells (reference 18 incorporated by reference). These authors demonstrated that anandamide-induced apoptosis was independent of cannabinoid receptors and was induced through vanilloid receptors. In the current study, we also observed that anandamide was effective at inducing apoptosis in lymphoid cell lines that were screened.

In the current study, we observed that THC was able to induce apoptosis in tumor cells not only in vitro, but also in vivo. Furthermore, THC was effective in reducing the tumor load, prolonging the mean survival tine of tumor-bearing mice, as well as curing a significant proportion of such mice. Because THC is immunosuppressive and EL-4 is an immunogenic tumor (refeernce 36) it is possible that the immunosuppressive effects of THC may have interfered with the host's antitumor immunity, which may account for a lower percentage of cures. Thus, further manipulations of the dose of THC that would induce significant apoptosis without causing significant suppression of antitumor immunity providing development of a treatment regimen that improves cure rate further.

The current study demonstrates that targeting CB2 receptors to induce apoptosis provides a novel approach to treating malignancies of the immune system. The advantage in using CB2 receptor agonists is that they do not exhibit psychoactive properties. Thus preferred compounds for use in the method have relatively low CB1 activity and more preferably have relatively low vanilloid receptor activity (eg. having greater than 5 times CB2 than vanniloid activity). Because CB2 receptors are expressed exclusively on immune cells, use of CB2 receptor agonists will not be toxic to non-immune cells.

The present invention will now be described by way of illustration only by reference to the following non-limiting examples, figures and sequences. Further embodiments falling within the scope of the invention will occur to those skilled in the art in the light of these.

### EXPERIMENTAL

Materials and Methods

Mice: Adult (6-8 weeks of age) female C57BL/6 mice were purchased from the National Institutes of Health, Bethesda, Md. The mice were housed in polyethylene cages and given rodent chow and water ad libitum. Mice were housed in rooms maintaining a temperature of 74±2° F. and on a 12-hour light/dark cycle.

Reagents: THC was obtained from the National Institute of Drug Abuse (Rockville, Md.) and was initially dissolved in dimethyl sulfoxide (DMSO; Sigma, St Louis, Mo.) to a concentration of 20 mM and stored at −20° C. THC was further diluted with tissue culture medium for in vitro studies and phosphate-buffered saline (PBS) for in vivo studies. SR141716A and SR144528 were obtained from Sanofi Recherche (Montpellier, France). HU-210, anandamide, WIN55212, and JWH-015 were obtained from Tocris Cookson (Ellisville, Mo.).

Cell lines: The murine lymphomas (EL-4 and LSA), the murine mastocytoma (P815), the murine melanoma (B16F10), Sup-T1, a T-lymphoblastic leukemia cell line developed from an 8-year-old male, Jurkat, an acute T-lymphoblastic leukemia cell line generated from a 14-year-old male, Molt-4, an acute T-lymphoblastic leukemia cell line established from a 19-year-old male, and human glioma U251 cell line were all maintained in RPMI 1640 medium (Gibco Laboratories, Grand Island, N.Y.) supplemented with 5% fetal calf serum (FCS), 10 mM HEPES, 1 mM glutamine, 40 μg/mL gentamicin sulfate, and 50 μM 2-mercaptoethanol. In assays examining the effect of cannabinoid agonists on tumor-cell viability and apoptosis, the concentration of FCS ranged from 0% to 5%.

Primary leukemic cells: Peripheral blood samples were obtained from 2 patients diagnosed with ALL. The samples were referred to as ALL no. 1 and ALL no. 2. ALL no. 1 was obtained from a male patient newly diagnosed with common acute lymphoblastic leukemia antigen (CALLA) (CD10)-positive non-B, non-T ALL. ALL no. 2 was obtained from a female patient newly diagnosed with terminal deoxynucleotidyl transferase (TdT)-positive T-cell ALL. Informed consent was obtained following institutional guidelines and approval was obtained from the institutional review board of Virginia Commonwealth University. Consent was provided according to the Declaration of Helsinki.

The content of the lymphoblasts was greater than 70% as determined by flow cytometric analysis. Mononuclear cells were isolated by Ficoll-Paque density gradient centrifugation. In this study, the samples were cryopreserved and stored in liquid nitrogen before use. Viability after thawing was determined by trypan blue dye exclusion and was greater than 90%.

Measurement of the effect of cannabinoid receptor aponistson tumor-cell viability in vitro: Tumor cells were adjusted to 1×10⁶ cells/mL in medium containing 5% FCS or serum-free medium. The cells (1×10⁶) were cultured in 24-well plates in 2 mL medium in the presence or absence of various concentrations of cannabinoid receptor agonists for 2 to 24 hours. Finally, the cells were harvested and washed twice in PBS, and the viable cell count was determined by trypan blue dye exclusion.

Detection of cannabinoid-induced apoptosis in vitro: Tumor cells (1×10⁶ cells/well) were cultured in 24 well plates in the presence or absence of various concentrations of THC or other cannabinoid receptor agonists for 2 to 24 hours, as described above. Next, the cells were harvested, washed twice in PBS, and analyzed for the induction of apoptosis using either the terminal deoxynucleotidyl transferase-mediated end labeling (TUNEL) method or annexin V/propidium iodide (PI) method, as described elsewhere (references 14,15 incorporated herein by reference). To detect apoptosis using the TUNEL method, we washed the cells twice with PBS and fixed them with 4% p-formaldehyde for 30 minutes at room temperature. The cells were next washed with PBS, permeabilized on ice for 2 minutes, and incubated with fluorescein isothiocyanate-dUTP and TdT (Boehringer Mannheim, Indianapolis, Ind.) for 1 hour at 37° C. and 5% CO₂. To detect apoptosis using the annexin V/PI method, we washed the cells twice with PBS and stained them with annexin V and PI for 20 minutes at room temperature. The cells were washed twice with PBS. The levels of apoptosis in both the TUNEL and annexin/PI assays were determined by measuring the fluorescence of the cells by flow cytometric analysis. Five thousand cells were analyzed per sample.

Measurement of tumor-cell viability and induction of apoptosis in vivo: Groups of 5 C57BL/6 mice were injected intraperitoneally (IP) with 1×10⁶ EL-4 tumor cells suspended in 0.2 mL PBS. The control mice received PBS alone. Ten days later, the mice were injected with various concentrations of THC (0, 1, 3, or 5 mg/kg EP). The mice were killed 24 hours later and the EL-4 tumor cells were harvested from the peritoneal cavity by injecting 5.0 mL PBS, followed by aspiration of the peritoneal fluid from the cavity. The contaminating red blood cells were removed with red blood lysing solution (Sigma), and the tumor cells were washed twice with PBS. The number of viable cells was determined by trypan blue dye exclusion, and apoptosis was determined using the TUNEL assay. The presence of tumor cells in the peritoneal cavity was confirmed by the ability of the cells to grow in vitro and by the phenotype (Thyl-, CD4-, CD8-).

Effect of THC on survival of EL-4-challenged mice: Groups of 8 C57BL/6 mice were injected EP with 1×10⁶ EL-4 tumor cells in a volume of 100 μL PBS. One day following tumor injection, the mice received daily IP injections for 14 days with 5 mg/kg THC in a volume of 500 μL PBS. Control mice received injections with the vehicle control. The mice were observed daily for signs of morbidity and were euthanized. Mice that survived for more than 60 days were rechallenged with live EL-4 cells (1×10⁶) and tested for their ability to reject tumor and survive.

RNA isolation and reverse transcriptase-polymerase chain reaction (RT-PCR): RNA was isolated from approximately 1×10⁷ cells using the RNeasy Mini Kit (Qiagen, Valencia, Calif.). Because CB1 and CB2 are encoded by single exons, a DNase digestion was included in the isolation procedure to limit the possibility of PCR amplification of CB1 and CB2 from genomic DNA. cDNA was prepared with the Qiagen OmniScript RT kit using 1 μg RNAas template for first-stand synthesis. Mouse and human CB1 was amplified using primers H CB1 U (5′-CGTGGGCAGCCTGTTCCTCA-3′) and H CB1 L (5′-CATGCGGGCTTGGTCTGG-3′), which yield a product of 403 bp. Human CB2 was amplified using primers H CB2 U (5′-CGCCG-GAAGCCCTCATACC-3′) and H CB2 L (5′-CCTCATTCGGGCCATTCCTG-3′), which yield a product of 522 bp. Mouse CB2 was amplified using M CB2 U (5′-CCGGAAAAGAGGATGGCAATGAAT-3′) and M CB2 L (5′-CTGCTGAGCGCCCTGGAGAAC-3′), which yield a product of 479 bp. β-Actin was used as a positive control, with primers M BA U(5′-AAGGCCAACCGTGAAAAGATGACC-3′) and M BAL(5′-ACCGCTCGTT GCCAATAGTGATGA-3′), with a product size of 427 bp. PCR reactions were carried out using the following parameters: 95° C. for 15 seconds, 58° C. for 15 seconds, and 72° C. for 30 seconds for 35 cycles; followed by a final 5 minutes at 72° C. in an Applied Biosystems GeneAmp 9700 (Foster City, Calif.). The resulting PCR products were separated on a 1% agarose gel.

Results:

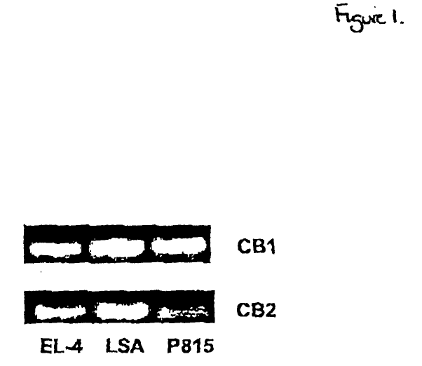

Expression of CB1 and CB2 receptors in EL-4, LSA, and P815 murine tumor cells: The expression of CB1 and CB2 cannabinoid receptor mRNA was determined by RT-PCR (FIG. 1). This analysis revealed that all 3 murine tumor-cell lines expressed both CB1 and CB2 mRNA.

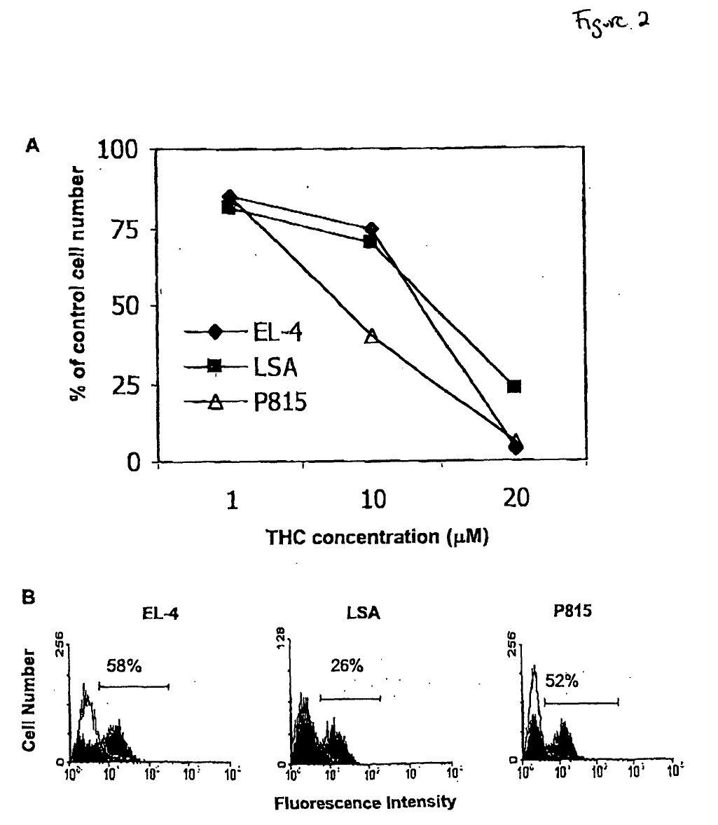

Exposure of EL-4, LSA, and P815 tumor cells to THC leads to a reduction in viability and induction of apoptosis in vitro: We examined whether THC exposure had an effect on the viability of EL-4, LSA, and P815 tumor cells in vitro. To this end, the tumor cells were cultured in medium containing 5% FCS and exposed to various concentrations of THC (0, 1, 10, and 20 μM) for 24 hours, and the viability was determined by trypan blue dye exclusion (FIG. 2A). The results showed that exposure to THC at concentrations of 10 μM or greater led to a significant reduction in the number of viable cells. Next, we analyzed the THC-treated tumor cells for induction of apoptosis by TUNEL staining (FIG. 2B).

The results demonstrated that THC induced significant apoptosis in all 3 cell lines in vitro. Together these results suggest that exposure of EL-4, LSA, and P815 tumor cells to THC in vitro led to significant cell killing by induction of apoptosis.

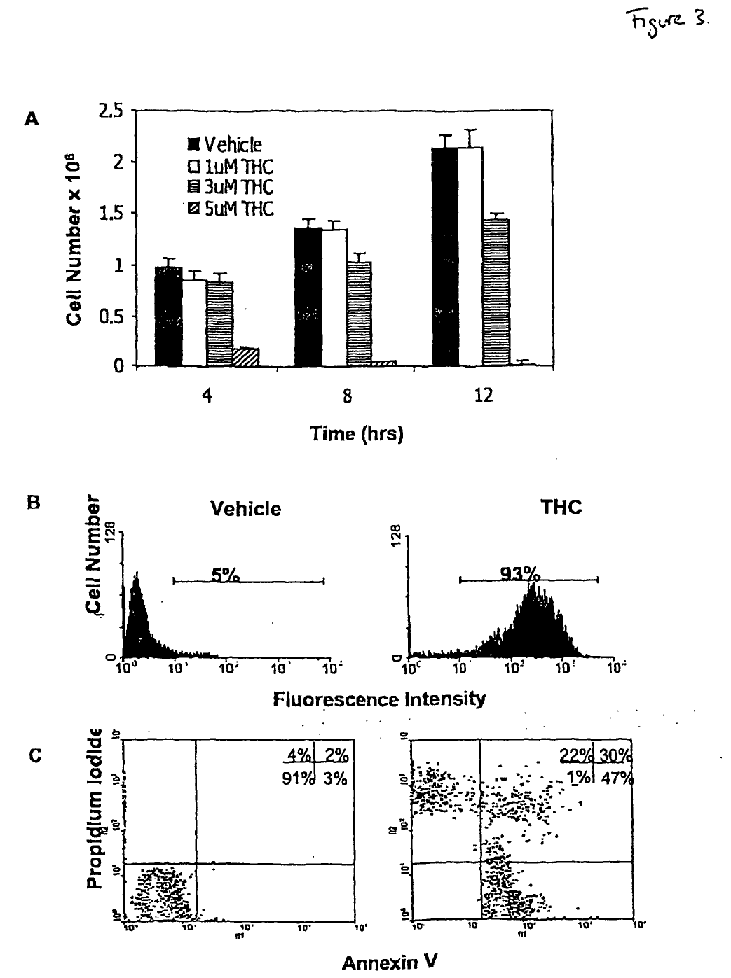

THC-induced effect on cellularity is dependent on exposure time and serum concentration: Previous studies suggested that the efficacy of THC may be directly related to the concentration in serum (reference 16, 17). Therefore, the inventors examined whether culturing tumor cells in serum-free medium would have an effect on THC-induced killing of tumor cells. This was accomplished by exposing EL-4 tumor cells to various concentrations of THC (1, 3, and 5 μM) or the vehicle for 4, 8, or 12 hours in serum-free medium and determining the cell viability. The results showed that by culturing the cells in serum-free medium, we dramatically reduced the concentration of THC needed to decrease tumor-cell viability (FIG. 3A). For example, exposure of EL-4 tumor cells to as low as 5 μM THC for 4 hours led to a significant decrease in tumor-cell viability (FIG. 3A) and an increase in the induction of apoptosis (FIG. 3B,C). Also, at 12 hours, THC at a concentration of 3 μM was able to cause a significant decrease in tumor-cell viability (FIG. 3A). The data shown in FIGS. 3B and 3C demonstrate that apoptosis induced by THC was evident using both the TUNEL and annexin/PI methods. Previous studies have shown that cells positive for annexin alone represent early apoptotic cells, whereas those positive for both annexin and PI are late apoptotic/necrotic cells, and cells positive for PI alone are necrotic cells (see reference 15). Thus, the majority of THC-treated cells appeared to be in an early or late apoptotic stage of death (FIG. 3C). It was also noted that in serum-free medium, the time required to induce tumor-cell killing was decreased significantly to 4 hours at a concentration of 51M THC (FIG. 3A). Because serum interfered with THC-induced apoptosis, all subsequent experiments were performed in serum-free medium.

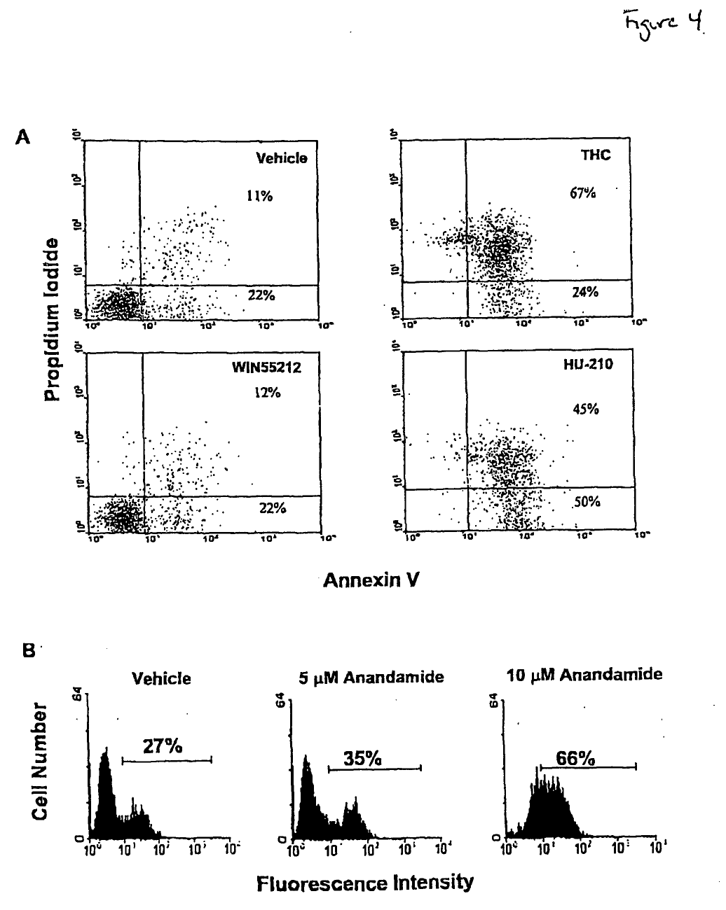

HU-210 and anandamide, but not WIN-55212 induce apoptosis in EL-4 tumor cells in vitro: Three additional cannabinoid receptor agonists were tested for their ability to induce apoptosis in EL-4 tumor cells. In FIG. 4A, EL-4 tumor cells were exposed to 3 μM THC, WIN55212, and HU-210 for 4 hours. The cells were then analyzed for apoptosis using the annexin/PI method (FIG. 4A). The results showed that exposure to THC or HU-210 led to a significant increase in apoptosis when compared with the controls. In contrast, exposure to 3 μM WIN55212 had no significant effect on the induction of apoptosis. In addition, we examined the effects of anandamide exposure on the induction of apoptosis (FIG. 4B). EL-4 tumor cells were exposed to 5 and 10 μM anandamide for 4 hours. The cells were analyzed for apoptosis using the TUNEL assay. The results shored that exposure to 5 μM led to a slight increase in apoptosis;

FIG. 1. The expression of CB1 and CB2 mRNA in EL-4, LSA, and P815 tumor cells. The expression of CB1 and CB2 mRNA was determined by RT-PCR analysis. Total RNA was isolated from EL-4, LSA, and P815 tumor cells. mRNA was reverse transcribed and amplified by PCR with primers specific for CB1 and CB2. A photograph of ethidium bromide-stained amplicons is depicted.

FIG. 2. Exposure of murine tumor cells of immune origin to THC in vitro leads to a reduction in cell viability and induction of apoptosis. (A) The effect of THC on tumor-cell viability was determined by culturing EL-4, LSA, and P815 tumor cells for 24 hours in medium containing 5% FCS in the presence of various concentrations of THC (1, 10, and 20 μM) or the vehicle. The viable cell number was determined by trypan blue dye exclusion. The data were expressed as percentage of control viable cell number. (B) The effect of THC on the induction of apoptosis in EL-4, LSA, and P815 tumor cells was determined by culturing the tumor cells for 24 hours in medium containing 5% FCS in the presence of 201M THC (filled histogram) or the vehicle (empty histogram). Apoptosis was quantified using the TUNEL method, and the cells were analyzed using a flow cytometer. Exposure to 10 μM anandamide led to significant levels of apoptosis.

THC treatment leads to reduced tumor burden and apoptosis in vivo: We examined whether treatment of tumor-bearing mice with THC was effective at killing tumor cells in vivo. To this end, C57BL/6 mice were injected with EL-4 tumor cells (1×10⁶).

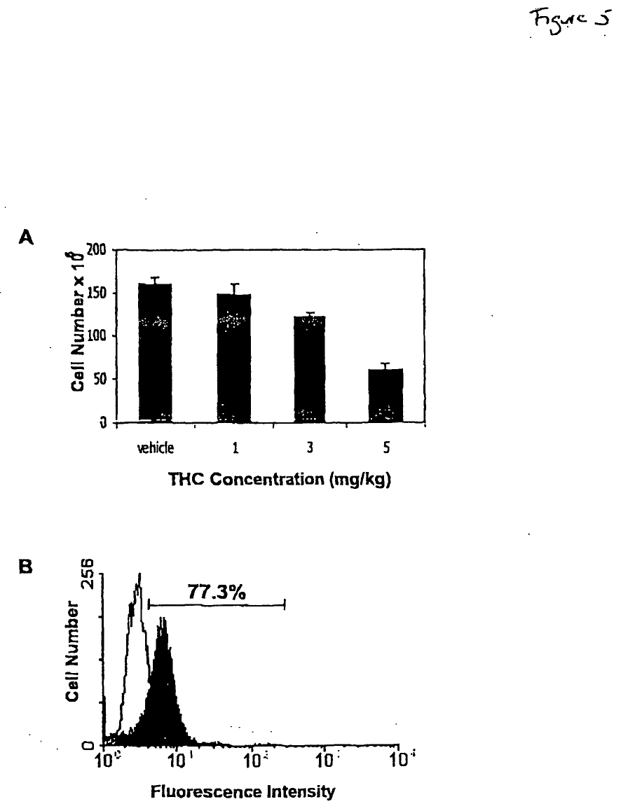

On day 10 of tumor growth, the mice were injected EP with various doses of THC (1, 3, or 5 mg/kg) or the vehicle. One day later, the mice were killed and injected with 5 mL PBS into the peritoneal cavity. The peritoneal fluid was aspirated and analyzed for viable tumor cells and for apoptosis. The data demonstrated that THC caused a dose-dependent decrease in the viable tumor-cell number found in the peritoneal cavity (FIG. 5A). THC failed to cause a decrease in cellularity at 1 mg/kg, but it was effective at 3 and 5 mg/kg.

Furthermore, when cells collected from mice treated with 5 mg/kg were analyzed for apoptosis, a significant proportion (77.3%) of the tumor cells showed apoptosis (FIG. 5B). These data suggest that THC was effective in vivo to induce apoptosis and kill the EL-4 tumor cells.

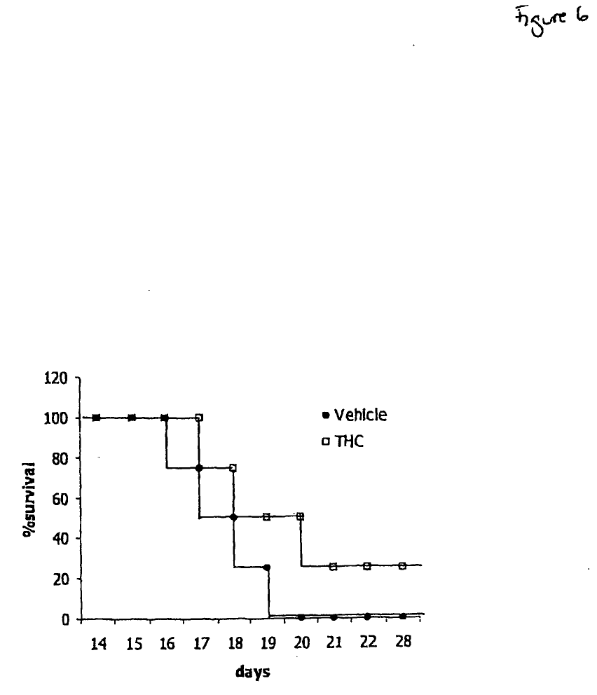

THC treatment can cure tumor-bearing mice: Next we tested whether THC treatment can cure EL-4 tumor bearing mice. To this end, mice were injected with EL-4 tumor cells (1×10⁶) and then given a daily injection of 5 mg/kg THC for 14 days. The mice were observed for survival and, upon exhibiting signs of morbidity, were immediately euthanized. The results showed that treatment with THC led to a significant increase in survival (FIG. 6). Interestingly, 25% of the mice survived the tumor challenge (FIG. 6). Also, they were completely cured in asmuch as they were resistant to rechallenge with the specific tumor (data not shown). Taken together, these results suggest that THC can exert anticancer properties in vivo.

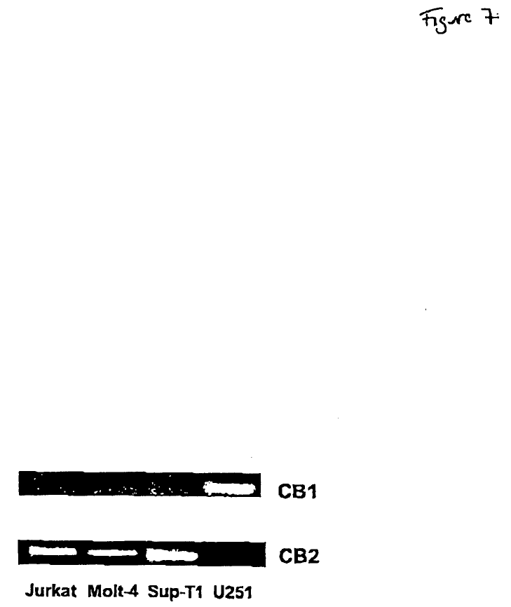

Expression of CB1 and CB2 cannabinoid receptors on human Molt-4, Jurkat, and Sup-T1 tumor-cell lines: Next, we tested whether human leukemia/lymphoma cell lines express cannabinoid receptors. The expression of CB1 and CB2 cannabinoid receptor mRNA was determined using RT-PCR analysis (FIG. 7). The results showed that all 3 cell lines screened expressed significant levels of CB2 mRNA. However, unlike in the murine tumor-cell lines, CB1 mRNA was not detected in these 3 cell lines. In these experiments, we used a human glioma cell line, U251, as a positive control for CB1 expression.

THC, HU-210, and anandamide induce apoptosis in human leukemia and lymphoma cell lines in vitro: Next, we examined whether exposure of human leukemia and lymphoma cell lines to THC or HU-210 would lead to induction of apoptosis. To this end, human tumor-cell lines Jurkat, Molt-4, and Sup-T1 were exposed to various concentrations of THC, HU-210 (2.5, 5, and 10 μM), or the vehicle for 4 hours, and the induction of apoptosis was determined using the TUNEL method. The results showed that exposure of the Jurkat, Molt-4, and Sup-T1 cell lines (FIG. 3).

THC is more effective in serum-free medium: (A) EL-4 tumor cells were cultured in serum-free medium in the presence of various concentrations of THC (1, 3, and 5 μM) or the vehicle for 4, 8, or 12 hours. The number of viable cells was determined by trypan blue dye exclusion The data represent the mean ±SEM of duplicate wells. (B) EL-4 tumor cells were cultured in serum-free medium in the presence of vehicle control (DMSO) or THC (5 μM) for 4 hours. The, level of apoptosis induction was determined using the TUNEL method. (C) EL-4 cells cultured with THC as described above were stained with annexin V/PI and analyzed using a flow cytometer FIG. 4.

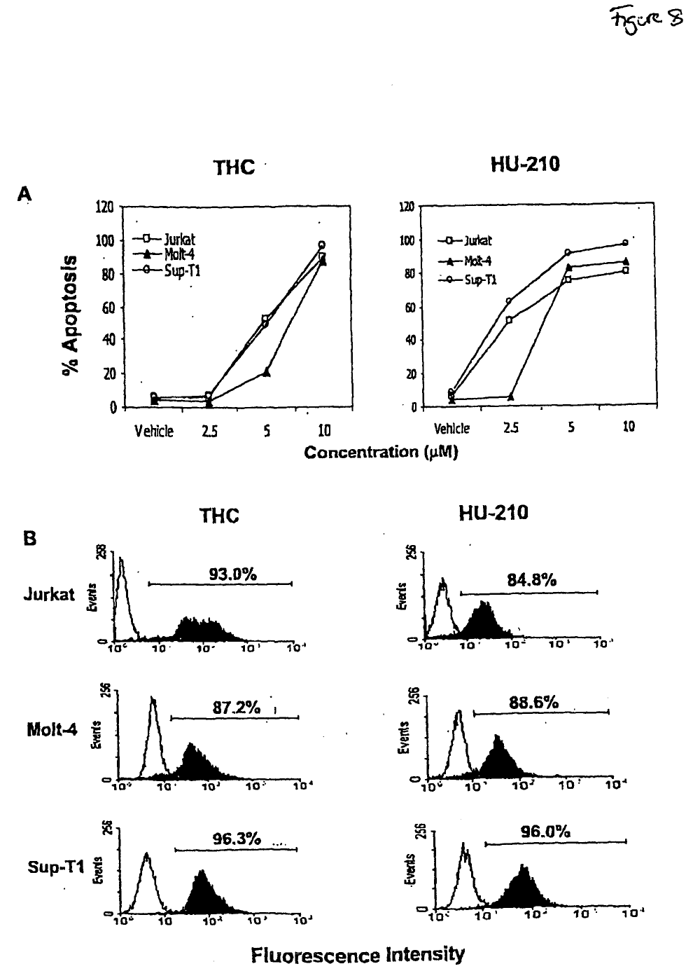

THC, HU-210, and anandamide, but not WIN55212, induce apoptosis in EL-4 tumor cells in vitro: (A) EL-4 tumor cells were cultured in serum-free medium for 4 hours in the presence of vehicle, THC (3 μM), WIN55212 (3 μM), and HU-210 (3 μM). The level of apoptosis was quantified by annexin/PI staining, as described in FIG. 3. (B) EL-4 tumor cells were cultured in serum-free medium for 4 hours in the presence of vehicle or anandamide (5 and 10 μM). The level of apoptosis was quantified by TUNEL assay to greater than or equal to 5 μM THC or HU-210 led to significant levels of apoptosis (FIG. 8A). THC at 10 μM and HU-210 at 511M concentrations caused greater than 80% apoptosis (FIG. 8A).

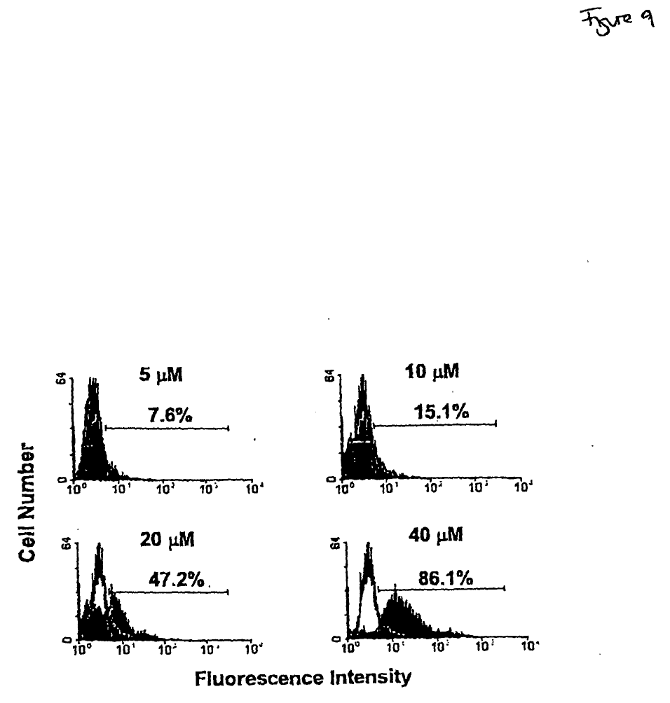

FIG. 8B shows a representative experiment using the TUNEL assay. In addition, we examined the effects of anandamide expo-sure on the induction of apoptosis in Molt-4 tumor cells. Molt-4 tumor cells were cultured for 4 hours in the absence or presence of various concentrations of anandamide (5, 10, 20, and 40 μM). The level of apoptosis was quantified using the TUNEL method (FIG. 9). The results showed that anandamide at concentrations of 20 μM or greater induced significant levels of apoptosis in Molt-4 tumor cells. Together, these data suggest that THC, HU-210, and anandamide can induce apoptosis in various human leukemia and lym-phoma cell lines.

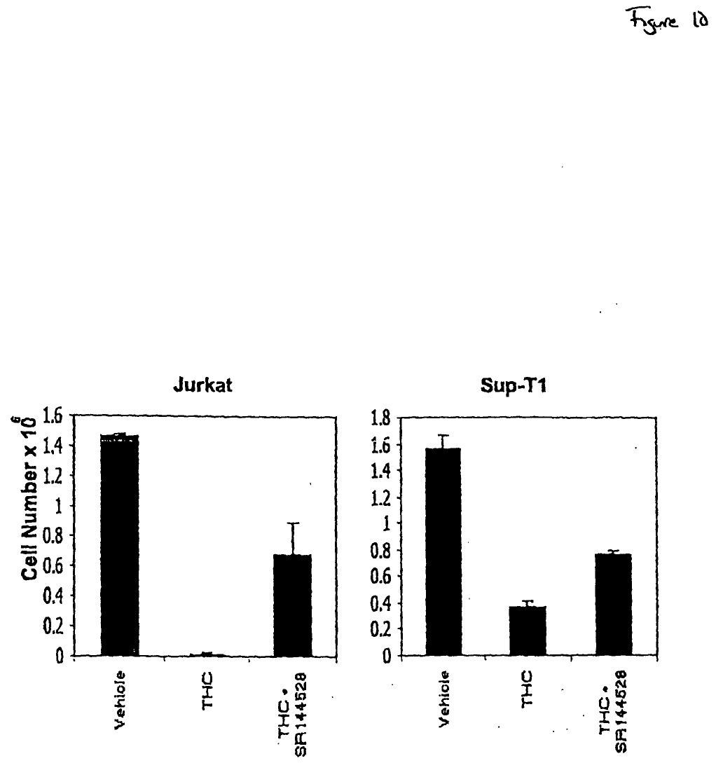

THC-induced reduction in viable cell number is mediated through the CB1 and CB2 cannabinoid receptors: Because the human tumor-cell lines screened exhibited CB2 but not CB1 receptors, we tested whether THC was acting through CB2 receptors to induce apoptosis. To this end, Jurkat and Sup-T1 cells were incubated with 5 μM THC in the presence of CB2 antagonists or the vehicle. After 4 hours, the viable cell number was determined by trypan blue dye exclusion (FIG. 10). The results showed that exposure to THC led to a dramatic reduction in the number of viable tumor cells. However, when the cells were cocultured with the CB2 antagonist, the viable cell numbers increased significantly, thereby reversing the effect of THC. Together, these results suggest that THC-induced reduction in viable cell number and increase in the induction of apoptosis were mediated through the CB2 cannabinoid receptors.

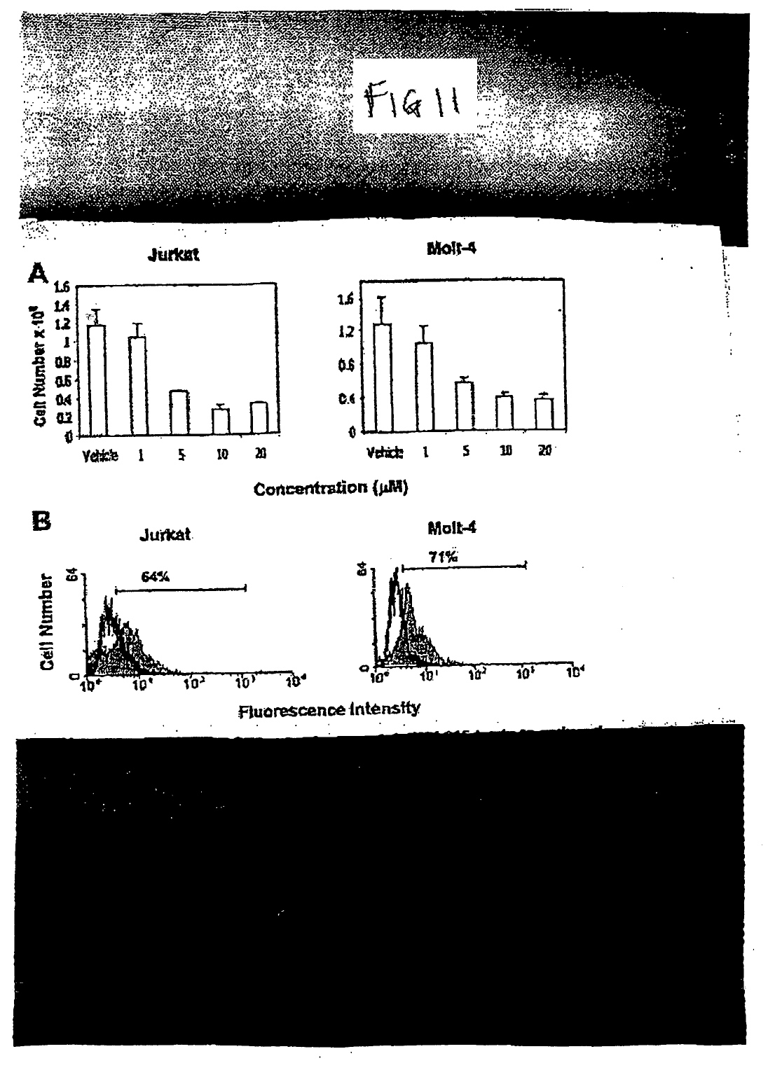

Exposure of Jurkat and Molt-4 tumor cells to CB2 receptor agonist, JWH-015, leads to a reduction in viability and induction of apoptosis in vitro: Next we examined whether exposure to a CB2-selective agonist would lead to tumor-cell death and induction of apoptosis. Jurkat and Molt-4 tumor cells were exposed to various concentrations of the CB2-selective agonist JWH-015 (1, 5, 10, and 20 μM) or the vehicle in serum-free medium for 24 hours. The results showed that exposure of Molt-4 and Jurkat tumor cells to 5 μM or greater concentrations of JWH-015 led to a significant decrease in the number of viable tumor cells (FIG. 11A). Next, we examined whether exposure to JWH-015 would lead to the induction of apoptosis. Jurkat and Molt-4 tumor cells were exposed to JWH-015 for 24 hours, and apoptosis was determined by TUNEL assay (FIG. 11B). The results showed that exposure of Jurkat and Molt-4 tumor cells to 5 M JWH-015 led to significant induction of apoptosis. Together these results suggest that treatment of Jurkat and Molt-4 tumor cells with CB2-selective agonist can lead to a significant reduction in cell viability and induction of apoptosis.

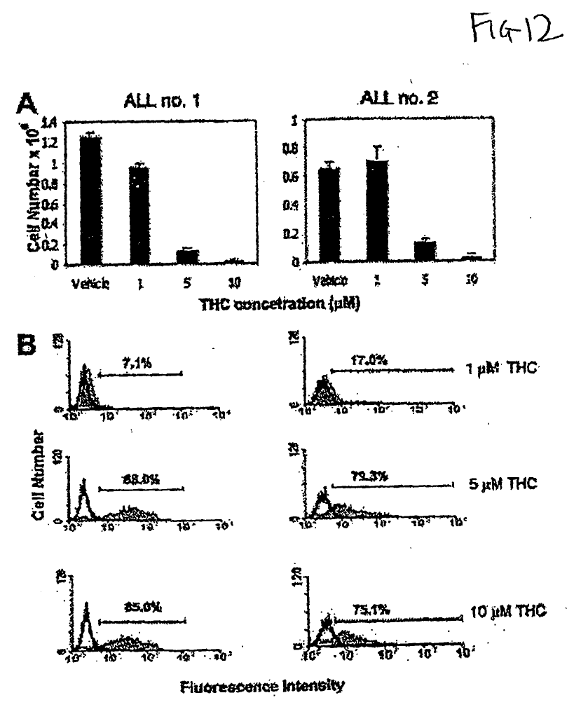

THC induces apoptosis in primary ALL cells in vitro: Next we examined whether exposure of primary ALL cells to THC would have any effect on tumor-cell viability or induction of apoptosis. To this end, lymphoblasts isolated from peripheral blood of 2 patients with ALL (ALL no. 1 and ALL no. 2) were cultured in the presence of various concentrations of THC (1, 5, and 10 μM) or vehicle (DMSO) for 2 hours. The viable cellularity was determined by trypan blue dye exclusion. The results showed that exposure of the ALL samples to 5 μM or greater concentrations of THC resulted in significant reduction in viability (FIG. 12A). In addition, we examined the effect of THC exposure on the induction of apoptosis using the TUNEL method and observed that exposure of cells from both ALL patients to 51M or greater concentrations of THC led to significant induction of apoptosis (FIG. 12B).

FIG. 5. THC treatment leads to reduced tumorburd en and tumor-cell apoptosis in vivo. C57BL/6 mice were injected IP on day 0 with 1×10⁶ EL-4 tumor cells. On day 10, the mice were treated with various doses of THC (1, 3, or 5 mg/kg IP) or the vehicle. One day later, the peritoneal cavity was flushed with 5 mL PBS, and the tumor cells were collected by aspiration. (A) The cell number was determined by trypan blue dye exclu-sion. The data represent the mean ±SEM from groups of 3 mice. (B) The tumor cells recovered from the peritoneal cavity were tested for apoptosis using the TUNEL method. Filled histogram shows tumor cells exposed to THC and open histogram shows cells exposed to the vehicle

FIG. 6. Treatment with T14C increases survival of EL-4 tumor-bearing mice: C57BL/6 mice (8 per group) were injected IP with 1×10⁶ EL-4 tumor cells on day 0. From day 1 onward, the mice were treated daily for 14 days with THC (5 mg/kg) or the vehicle control by the IP route. The mice were observed daily for survival and signs of morbidity. The data depicted are representative of 3 separate experiments.

FIG. 7. The expression of CB1 and CB2 mRNA in Molt-4 Jurkat. Sup-T1, and U251 human tumor cells: The expression of CB1 and CB2 was determined by RT-PCR analysis. Total RNA was isolated from Molt-4, Jurkat, Sup-T1, and U251 tumor cells. mRNA was reverse transcribed and amplified by PCR with primers specific for CB1 and CB2. A photograph of ethidium bromide-stained amplicons is depicted. These results were further corroborated by staining the ALL cells with annexin V and PI. Together, these results suggest that exposure of primary ALL cells to THC can lead to significant tumor killing mediated by the induction of apoptosis.

FIG. 8. THC and HU-210 exposure leads to the induction of apoptosis in human lymphoid tumors in vitro. Human tumors Molt-4, Jurkat, and Sup-T1 were cultured in serum-free medium in the presence or absence of various concentrations of THC, HU-210 (2.5, 5, and 10 μM), or the vehicle for 4 hours. (A) The induction of apoptosis was determined by the TUNEL method, and the percentage of apoptotic cells was plotted. (B) A representative experiment in which human tumor cells cultured with 10 μM of THC or HU-210 (filled histograms) or the vehicle (open histograms) were analyzed for apoptosis using TUNEL assay.

FIG. 9. Anandamide exposure leads to the induction of apoptosis in Molt-4 tumor cells in vitro. Molt-4 tumor cells were cultured in serum-free medium in the presence or absence of various concentrations of anandamide (5, 10, 20, and 40 μM) or the vehicle for 4 hours. The induction of apoptosis was determined by the TUNEL method. A representative experiment in which Molt-4 tumor cells were cultured with anandamide (filled histogram) or the vehicle (open histogram) is depicted.

FIG. 10. CB2 receptor antagonists can reverse the toxicity of THC. Jurkat and Sup-T1 human tumor cells were cultured for 4 hours in the presence of THC (5 μM) or the vehicle. In addition, the cultures received the CB2 antagonist (5 μM). The viable cell number was determined by trypan blue dye exclusion. The data represent the mean # SEM of triplicate cultures.

FIG. 11. Exposure to the CB2-selective agonist JWH-015 leads to reduced cell viability and induction of apoptosis in Jurkat and Molt-4 tumor cells in vitro: (A) The effect of JWH-015 on tumor-cell viability was determined by culturing Jurkat and Molt-4 tumor cells for 24 hours in serum-free medium in the presence of various concentrations of JWH-015 (1, 5, 10, and 20 μM) or the vehicle. The viable cell number was determined by trypan blue dye exclusion. (B) The effect of JWH-015 on the induction of apoptosis in Jurkat and Molt-4 tenor cells was determined by culturing the tumor cells for 24 hours in serum-free medium in the presence of 5 μM JWH-015 (filled histogram) or the vehicle (empty histogram). Apoptosis was quantified using the TUNEL method, and the cells were analyzed using a flow cytometer.

FIG. 12. THC induces apoptosis in primary ALL cells in vitro: (A) The effect of THC on primary ALL cell viability was determined by culturing the cells for 2 hours in serum-fee medium in the presence of various concentrations of THC (1, 5, and 10 μM) or the vehicle. The viable cell number was determined by trypan blue dye exclusion. (B) The effect of THC on the induction of apoptosis in primary ALL cells was determined by TUNEL assay, as described in FIG. 2. Tumor cells were cultured as described above with THC (filled histogram) or the vehicle (empty histogram).

Apoptosis was quantified using the TUNEL method, and the cells were analyzed using a flow cytometer. The percentage of apoptotic cells following THC exposure is depicted in each histogram.

### REFERENCES

1. Berdyshev E V. Cannabinoid receptors and the regulation of immune response. Chem Phys Lip-ids. 2000; 108:169-190.

2. Watson S J, Benson J A Jr, Joy J E. Marijuana and medicine. assessing the science base: a summary of the 1999 Institute of Medicine report. Arch Gen Psychiatry. 2000;57:547-552.

3. Devane W A, Dysarz F A 3rd, Johnson M R, Melvin L S, Howlett A C. Determination and characteriza-tion of a cannabinoid receptor in rat brain. Mol Pharmacol. 1988;34:605-613.

4. Munro S, Thomas K L, Abu-Shaar M. Molecular characterization of a peripheral receptor for can-nabinoids Nature. 1993;365:61-65.

5. Devane W A, Hanus L, Breuer A, et al. Isolation and structure of a brain constituent that binds to the cannabinoid receptor. Science. 1992;258:1946-1949.

6. Felder C C, Briley E M, Axelrod J. Simpson J T, Mackie K, Devane W A. Anandamide, an endogenous cannabimimetic eicosanoid, binds to the cloned human cannabinoid receptor and stimulates receptor-mediated signal transduction. Proc Natl Acad Sci USA. 1993;90:7656-7660.

7. Mechoulam R, Ben-Shabat S, Hanus L, et al. Identification of an endogenous 2-monoglyceride, present in canine gut, that binds to cannabinoid receptors. Biochem Pharmacol. 1995;50:83-90.

8. De Petrocellis L, Melck D, Palmisano A, et al. The endogenous cannabinoid anandamide inhibits human breast cancer cell proliferation. Proc Natl Acad Sci U S A. 1998;95:8375-8380.

9. Sanchez C, Galve-Roperh I, Canova C, Brachet P, Guzman M. Delta9-tetrahydrocannabinol induces apoptosis in C6 glioma cells. FEBS Lett 1998;436:6-10.

10. Ruiz L, Miguel A, Diaz-Laviada I. Delta9-tetrahy-drocannabinol induces apoptosis in human prostate PC-3 cells via a receptor-independent mechanism. FEBS Lett. 1999;458:400-404.

11. Campbell V A. Tetrahydrocannabinol-induced apo-ptosis of cultured cortical neurones is associated with cytochrome c release and caspase-3 activation. Neuropharmacology. 2001;40:702-709.

12. Galve-Roperh I, Sanchez C, Cortes M L, del Pul-gar T G, Izquierdo M, Guzman M. Anti-tumoral action of cannabinoids: involvement of sustained ceramide accumulation and extracellular signal-regulated kinase activation. Nat Med. 2000;6: 313-319.

13. Sanchez C, de Ceballos M L, del Pulgar T G, et al. Inhibition of glioma growth in vivo by selective activation of the cb(2) cannabinoid receptor. Cancer Res. 2001;61:5784-5789.

14. Kamath A B, Xu H, Nagarkatti P S, Nagarkatti M. Evidence for the induction of apoptosis in thymocytes by 2,3,7,8- tetrachlorodibenzo-p-dioxin in vivo. Toxicol Appl Pharmacol. 1997;142:367-377.

15. Chen D, McKallip R J, Zeytun A, et al. CD44-defi-cient mice exhibit enhanced hepatitis after con-canavalin A injection: evidence for involvement of CD44 in activation-induced cell death. J. Immunol. 2001;166:5889-5897.

16. Zheng Z M, Specter S, Friedman H. Serum pro-teins affect the inhibition by delta-tetrabydrocan-nabinol of tumor necrosis factor alpha production by mouse macrophages. Adv Exp Med Biol. 1993;335:89-93.

17. Jacobsson S O, Rongard E, Stridh M, Tiger G, Fowler C J. Serum-dependent effects of tamoxifen and cannabinoids upon C6 glioma cell viability. Biochem Pharmacol. 2000;60:1807-113.

18. Maccarrone M, Lorenzon T, Bari M, Melino G, Finazzi-Agro A. Anandamide induces apoptosis in human cells via vanilloid receptors: evidence for a protective role of cannabinoid receptors. J Biol Chem. 2000;275:31938-31945.

19. Sarker K P, Obara S. Nakata M, Kitajima I, Ma-ruyama I. Anandamide induces apoptosis of PC-12 cells: involvement of superoxide and caspase-3. FEBS Lett. 2000;472:39-44.

20. Pertwec R G. Pharmacology of cannabinoid re-ceptor ligands. Curr Med Chem. 1999;6:635-664.

21. Landsman R S, Burkey T H, Consroe P, Roeske W R, Yamamura H I. SR141716A is an inverse agonist at the human cannabinoid CB1 receptor. Bur J. Pharmacol. 1997334:R1-R2.

22. Portier M, Rinaldi-Carmona M, Pecceu F, et al. SR 144528, an antagonist for the peripheral cannabinoid receptor that behaves as an inverse agonist. J Phar-macol Exp Ther. 1999;288:582-589.

23. Klein T W, Lane B, Newton C A, Friedman H. The cannabinoid system and cytokine network. Proc Soc Exp Biol Med. 2000;225:1-8.

24. Koh W S, Crawford R B, Kaminski N E. Inhibition of protein kinase A and cyclic AMP response ele-ment (CRE)-specific transcription factor binding by delta9-tetrahydrocannabinol (delta9-THC); a putative mechanism of cannabinoid-induced im-mune modulation. Biochem Pharmacol. 1997;53:1477-1484.

25. Schatz A R, Lee M, Condie R B, Pulaski J T, Kaminski N E. Cannabinoid receptors CB1 and CB2: a characterization of expression and adenylate cyclase modulation within the immune system. Toxicol Appl Pharmacol. 1997;142:278-287.

26. Herring A C, Koh W S, Kaminski N E. Inhibition of the cyclic AMP signaling cascade and nuclear factor binding to CRE and kappaB elements by cannabinol, a minimally CNS-active cannabinoid. Biochem Pharmacol. 1998;55.1013-1023.

27. Novak T J, Chen D, Rothenberg E V. Interleukin-1 synergy with phorphoinositide pathway agonists for induction of interleukin-2 gene expression: molecular basis of costimulation. Mol Cell Biol. 1990; 10:6325-6334.

28. Lenardo M J. Interleukin-2 programs mouse alpha beta T lymphocytes for apoptosis. Nature. 1991; 353:858-861.

29. Deng G, Podack E R. Suppression of apoptosis in a cytotoxic T-cell line by interleukin 2-mediated gene transcription and deregulated expression of the protooncogene bcl-2. Proc Natl Acad Sci USA. 1993;90:2189-2193.

30. Zhang X, Giangreco L, Broome H E, Dargan C M, Swain S L. Control of CD4 effector fate: transforming growth factor beta 1 and interleukin 2 syner-gize to prevent apoptosis and promote effector expansion. J Exp Med. 1995; 182:699-709.

31. Nagarkatti M, Hassuneh M, Seth A, Manicka-sundari K, Nagarkatti P S. Constitutive activation of the interleukin 2 gene in the induction of spon-taneous in vitro transformation and tumorigenicity of T cells. Proc Natl Acad Sci USA. 1994;91:7638-7642.

32. Hassuneh M R, Nagarkatti P S, Nagarkatti M. Evi-dence for the participation of interleukin-2 (IL-2) and IL-4 in the regulation of autonomous growth and tumorigenesis of Transformed cells of lym-phoid origin. Blood. 1997;89:610-620.

33. Pertwee R G. Pharmacology of cannabinoid CB1 and CB2 receptors. Pharmacol Ther. 1997;74:129-180.

34. Klein T W, Newton C, Friedman H. Cannabinoid receptors and immunity. Immunol Today. 1998; 19:373-381.

35. Chan P C, Sills R C, Braun A G, Haseman J K, Bucher J R. Toxicity and carcinogenicity of delta 9-tetrahydro-cannabinol in Fischer rats and B6C3F1 mice. Fun-dam Appl Toxicol. 1996;30:109-117.

36. Iezzi G, Rivolta L, Ronchetti A, et al. The immunoge-nicity of experimental tumors is strongly biased by the expression of dominant viral cytotoxic T-lymphocyte epitopes. Cancer Res. 1997;57:2564-2568.

1. A method of treating a patient in need of therapy for an abnormality of cells of the immune system comprising administration of a therapeutically effective dose of a compound having CB2 cannabinoid receptor activity.

2. A method as claimed in claim 1 wherein the abnormality is selected from the group consisting of malignancies of the immune system, an autoimmune disease, septic shock, transplantation reaction and allergy.

3. A method as claimed in claim 1 wherein the abnormality is selected from the group consisting of leukemias and lymphomas.

4. A method as claimed in claim 1 wherein the abnormality is primary acute lymphoblastic leukemia (ALL).

5. A method as claimed in any claim 1 wherein the compound is a CB2 agonist that has reduced psychotrophic activity as compared with THC.

6. A method as claimed in claim 1 wherein the selective CB2 agonist is selected from those having an affinity for CB2 receptors that is at least five times that for CB1.

7. A method as claimed in claim 6 wherein the selective CB2 agonist is selected from those having an affinity for CB2 receptors that is at least 10 times that for CB1.

8. A method as claimed in claim 6 wherein selective CB2 agonist is selected from those having an affinity for CB2 receptors that is at least 20 times that for CB1.

9. A method as claimed in claim 6 wherein selective CB2 agonist is selected from those having an affinity for CB2 receptors that is at least 100 times that for CB1.

10. Use of a compound of the first aspect of the invention for the manufacture of a medicament for the treatment of abnormalities of the immune system, particularly malignancies of the immune system, such as leukemia and lymphoma.

11. A pharmaceutical composition for treating abnormalities of the immune system comprising as active agent a CB2 agonist.
